(19) United States
(12) Patent Application Publication
(10) Pub. No.: US 2024/0087683 A1
(43) Pub. Date: Mar. 14, 2024

(54) CLASSIFICATION USING A MACHINE LEARNING MODEL TRAINED WITH TRIPLET LOSS

(71) Applicant: MICROSOFT TECHNOLOGY LICENSING, LLC, Redmond, WA (US)

(72) Inventors: Pramod Kumar SHARMA, Seattle, WA (US); Andy Daniel MARTINEZ, Pembroke Pines, FL (US); Liang DU, Redmond, WA (US); Robin ABRAHAM, Redmond, WA (US); Saurabh Chandrakant THAKUR, Bellevue, WA (US)

(21) Appl. No.: 17/932,212

(22) Filed: Sep. 14, 2022

(51) Int. Cl. G16B 40/00; C12Q 1/689; G06N 3/08

(52) U.S. Cl. CPC G16B 40/00; C12Q 1/689; G06N 3/084

(57) ABSTRACT

A machine learning model trained with a triplet loss function classifies input strings into one of multiple hierarchical categories. The machine learning model is pre-trained using masking language modeling on a corpus of unlabeled strings. The machine learning module includes an attention-based bi-directional transformer layer. Following initial training, the machine learning model is refined by additional training with a loss function that includes cross-entropy loss and triplet loss. This provides a deep learning solution to classify input strings into one or more hierarchical categories. Embeddings generated from inputs to the machine learning model capture language similarities that can be visualized in a cartesian plane where strings with similar meanings are grouped together.

### BACKGROUND

Artificial intelligence and machine learning models can be used to solve many problems better than conventional programming. Training creates a model that can be used to classify new items based on similarity to items used for training the model. For example, a machine learning model may be trained on images of dogs and cats then used to classify a new image as either a dog or a cat. Thus, the training dataset and the techniques for training a model are key to creating the functionality of that model.

Natural language processing (NLP) is a branch of artificial intelligence concerned with enabling computers to understand text and spoken words. NLP combines computational linguistics—rule-based modeling of human language—with statistical, machine learning, and deep learning models. NLP models have been based on convolutional neural networks (CNNs) and recurrent neural networks (RNNs). More recently, a type of deep learning model called a “transformer” has been used for natural language processing and produced better results than CNNs and RNNs. A transformer uses the mechanism of self-attention to differentially weigh the significance of each part of input data. Pretrained systems have been created by training transformers with large language datasets. One example is BERT (Bidirectional Encoder Representations from Transformers) that was trained with a large corpus of English-language text. A pretrained model can then be used for a number of tasks.

The field of artificial intelligence and machine learning provides flexibility and offers the possibility of improved solutions for NLP and other fields. It is with respect to these and other considerations that the disclosure made herein is presented.

### SUMMARY

The techniques disclosed herein use a machine learning model to classify an input sequence string according to a hierarchical taxonomy. The input sequence string may be any string of data such as words in a natural language, a string of numbers, or a string of letters. First, the machine learning model is pre-trained to gain an understanding of patterns within the sequence strings. The machine learning model learns the intra-string patterns by masked language modeling which is an example of autoencoding language modeling. Input tokens (e.g., words in a natural language sentence) in the hidden sequence strings are and the model is trained to correctly predict the missing token. The language model maps the input sequence strings to embedding vectors that encode what the language model has learned about the sequence strings.

Then, the pre-trained model is fine-tuned to correctly classify a new input sequence string into a position within a taxonomic hierarchy. This stage of training is performed using a cross-entropy loss function. The model is also trained using a triplet loss function so that similar sequence strings are represented by high-dimensional vectors that are located close to each other in a shared embedding space. The results of the cross-entropy loss function and the triplet loss function are combined to generate a loss value that is applied during back-propagation. For example, the cross-entropy loss function and the triplet loss function may be summed to arrive at a composite loss value.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 illustrates using a machine learning model to infer a taxonomic classification of a sequence string.

FIG. 2 illustrates training a machine learning model for taxonomy classification of a raw sequences.

FIG. 3 illustrates pre-training a language model with masking language modeling to map a string of letters to a vector of numbers.

FIG. 4 illustrates converting text-based strings into a vector of numeric values that can be processed by a model.

FIG. 5 illustrates converting a taxonomy location into a numeric representation.

FIG. 6 illustrates a similarity comparison engine for determining if two taxonomy locations are similar.

FIG. 7A illustrates generating DNA sequence triplets with which to train a DNA classifier model.

FIG. 7B illustrates a byte pair encoded tokenizer.

FIG. 7C illustrates a language model inferring embedding vectors from tokens.

FIG. 7D illustrates providing embedding vectors to additional fully-connected layers of the classifier model.

FIG. 7E illustrates providing taxonomy location predictions, along with the corresponding expected taxonomy locations, to a cross-entropy loss function.

FIG. 8 illustrates applying a triplet loss function to embedding vectors.

FIG. 9 illustrates combining the results of cross-entropy loss functions and triplet loss functions into a composite loss.

FIG. 10A illustrates a 2D depiction of embedding vectors shaded by domain after dimensionality reduction.

FIG. 10B illustrates a 2D depiction of embedding vectors shaded by phylum after dimensionality reduction.

FIG. 11 is a flow diagram of an example method for classifying a sequence within a taxonomic hierarchy.

FIG. 12 is a computer architecture diagram illustrating an illustrative computer hardware and software architecture for a computing system capable of implementing aspects of the techniques and technologies presented herein.

FIG. 13 is a diagram illustrating a distributed computing environment capable of implementing aspects of the techniques and technologies presented herein.

### DETAILED DESCRIPTION

Metagenome mapping involves extraction and identification of all genomic sequences from environmental samples. Environmental samples, such as soil samples, food samples, or biological tissue samples can contain extremely large numbers of organisms. For example, it is estimated that the human body, which relies upon bacteria for modulation of digestive, endocrine, and immune functions, can contain up to 100 trillion organisms. Advances in sequencing and screening technologies have made it possible to obtain polynucleotide (i.e., DNA or RNA) sequences of the organisms in an environmental sample.

The polynucleotide sequences are analyzed to determine which species of organisms are found in a given sample and potentially identify previously unknown species. Determining what species are represented in the polynucleotide sequence in a sample may help with a crime scene investigation, monitoring sewage for disease, or better understanding the biodiversity of a pond. Typically, sequences from a sample are compared to known sequences using a sequence alignment algorithm such as BLAST (Basic Local Alignment Search Tool) to find a match with a reference database of sequence. Use of BLAST in metagenomic analysis is discussed in Bazinet A L, Ondov B D, Sommer D D, Ratnayake S. BLAST-based validation of metagenomic sequence assignments. PeerJ. 2018 May 28; 6:e4892. BLAST is widely considered the “gold standard” for sequence comparison, although it is computationally intensive, making it infeasible to run on the millions of reads typically generated by metagenomic sequencing studies. BLAST and other sequence comparison algorithms are also inflexible in their application meaning. The standard used to determine a “match” is hard-coded by the algorithm and cannot adapt to the type of organisms included in a reference databases. The algorithm employed by BLAST leverages human understanding of biology. There is no easy mechanism to update or modify the algorithm. Moreover, the human understanding used to create the algorithm may not be accurate or complete.

Additional techniques for metagenomic analysis that are faster and/or more flexible than comparison algorithms would have utility for analysis of environmental samples of DNA and RNA. The results of such analysis could be used to modify treatment regimens for patients, guide environmental remediation efforts, improve crop production, identify the source of forensic samples, and many other applications.

To address the above and other issues, a machine learning model is used to determine where a DNA sequence is located within a taxonomy. First, a language model is pre-trained with a corpus of DNA sequences in order to gain an understanding of patterns within DNA. For example, DNA sequences may be represented by strings of letters, and an unsupervised masked-language modeling technique may be applied to learn about patterns in the letters. The language model may be pre-trained to output an embedding vector for a given DNA sequence. An embedding vector is an array of numbers that encode what the language model has learned about a particular DNA sequence. Embedding vectors that are closer together, as measured by a Euclidian distance, are considered more closely related than embedding vectors that are further apart from each other.

The pre-trained language model is one component of a larger machine learning model used to predict where a DNA sequence is located within a taxonomy. In some configurations, the model as a whole is trained with triplets of DNA sequences selected from annotated DNA sequence training data. Each sequence in the training data is annotated with a location in the taxonomy. A DNA sequence triplet is created such that two of the sequences are similar and two of the sequences are dissimilar. Specifically, a DNA sequence triplet is selected to have an anchor sequence, a “positive” sequence that is taxonomically similar to the anchor sequence, and a “negative” sequence that is taxonomically dissimilar to the anchor sequence. The anchor sequence may be selected at random, or the anchor sequence may be selected based on inclusion in a particular hierarchy of the taxonomy, or any other criteria. Sequences that are taxonomically similar and dissimilar are selected in order to train the model to cluster similar sequences while distancing dissimilar sequences, where similarity reflects sharing at least a defined number of ancestors in the taxonomy.

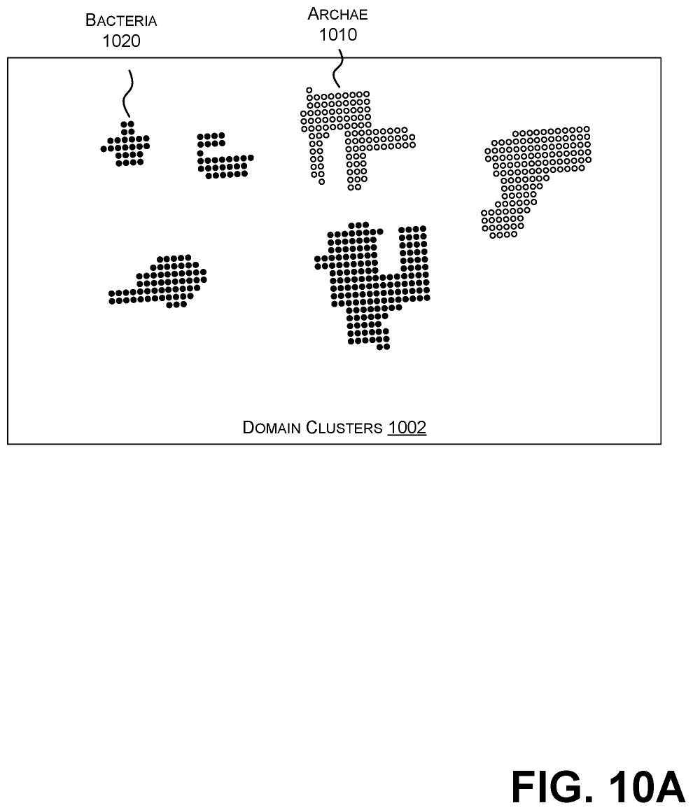

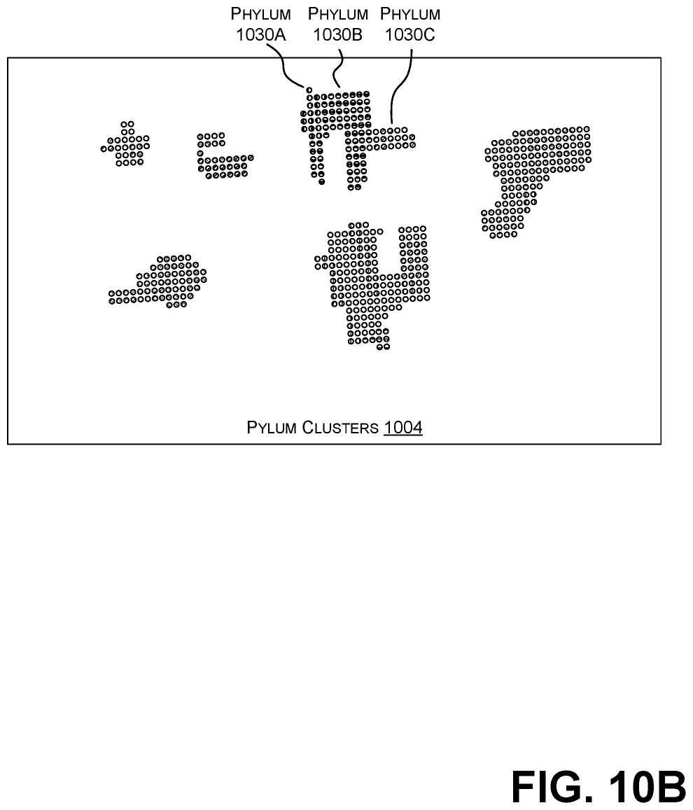

Each sequence of a DNA sequence triplet may be provided in turn to the pre-trained language model. The resulting embedding vectors constitute an embedding vector triplet, one embedding for the anchor sequence, one embedding for the positive sequence (i.e., the taxonomically similar sequence), and one embedding for the negative sequence (i.e., taxonomically dissimilar sequence). The embedding vector triplet is evaluated by a triplet loss function, the result of which trains the model to cluster embedding vectors of similar sequences while distancing embedding vectors of dissimilar sequences. By defining similarity as sharing at least a defined number of ancestors in the taxonomy, the triplet loss function trains the model to cluster embedding vectors that are close together in the taxonomy. FIGS. 10A and 10B visualize clusters generated in this way.

In addition to training the model to cluster similar DNA sequences, the model may also be trained at the same time to accurately predict the location of a DNA sequence within the taxonomy. To do this, each embedding vector of the triplet of embedding vectors is provided to additional layers of the larger machine learning model, yielding a triplet of taxonomy location predictions. In some configurations, taxonomy locations are encoded as single integer values, although arrays of values or other encodings are similarly contemplated.

Each of the taxonomy location predictions may be compared to an expected location defined in the taxonomy-annotated training data. For example, a cross-entropy loss function may evaluate whether the predicted location was the expected location. The resulting values may be added together into a total cross-entropy loss. The total cross-entropy loss may then be added to the triplet loss discussed above. This composite loss is then used to train the model. In some configurations, the model is trained by performing backpropagation on the additional layers as well as the pre-trained language model.

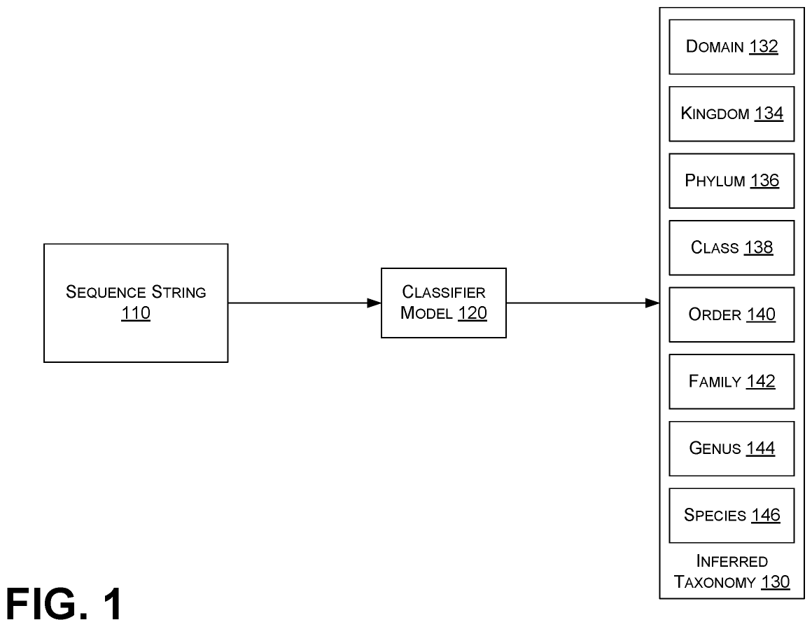

FIG. 1 illustrates using a classifier model 120, which is a type of machine learning model, to infer a taxonomy 130 of a DNA sequence string 110. The classifier model 120 may also be referred to as a DNA classifier model 120. The DNA sequence may be a subsequence of DNA, such as a 16s RNA gene sequence that is known in biology to be useful for reconstructing phylogenies. One taxonomy commonly used in biology is a hierarchy of domain 132, kingdom 134, phylum 136, class 138, order 140, family 142, genus 144, and species 146. For example, an input DNA sequence “CGTAGAGCG . . . ” could be inferred as belonging to the domain “bacteria”, the phylum “Abditibacteriota”, the class “Abditibacteria”, the order “Abditibacteriales”, the family “Abditibacteriaceae”, the genus “Abditibacterium”, and the species “Abditibacterium unsteinense”.

One application for the DNA classifier model 120 is in metagenomic analysis. Metagenomics is the study of genetic material recovered directly from environmental samples. The term metagenome references the idea that a collection of genes sequenced from the environment could be analyzed in a way analogous to the study of a single genome. Metagenomics has been defined as the application of modern genomics technique without the need for isolation and lab cultivation of individual species. Thus, the input to the DNA classifier model 120 may be sequence data obtained from sequencing genomic material collected from an environmental sample.

The sequence data may contain a large number of unique reads from a plurality of organisms. There may be thousands, hundreds of thousands, millions, hundreds of millions, or more reads provided to the DNA classifier model 120. The environmental sample may be collected from any natural, artificial, or biological environment. For example, the environmental sample may be collected from a body of water, soil, or the digestive tract of a vertebrate such as a human.

The DNA classifier model 120 is a machine learning model that generates an embedding for reads provided in the sequence data. The embeddings are placed in an embedding space that groups phylogenetically similar organisms together. Distances between vectors representing the DNA sequences from the environmental sample and vectors representing known organisms are used to predict taxonomic classifications for the sequence data from the environmental sample.

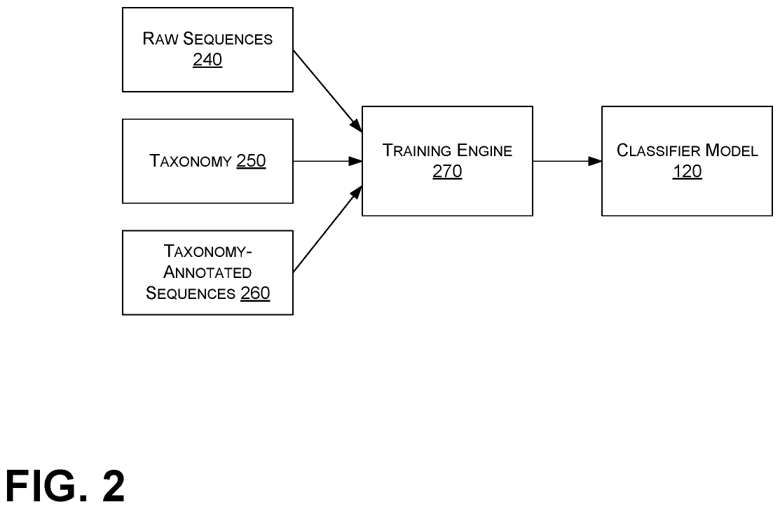

FIG. 2 illustrates training a machine learning model for taxonomy classification of a DNA sequence. In some configurations, training engine 270 creates and refines classifier model 120 using an unannotated dataset of raw DNA sequences 240, taxonomy 250, and taxonomy-annotated sequences 260. The taxonomy-annotated sequences may also be referred to as taxonomy-annotated DNA sequences 260.

Unannotated raw DNA sequences 240 may be in the form of a string of characters, each of which represents one of the nucleotide bases adenine (A), cytosine (C), guanine (G), and thymine (T). While DNA sequences are referenced throughout this document, other sequences are similarly contemplated, including RNA sequences, proteins, amino acid sequences, and the like. Furthermore, other means of encoding raw DNA sequences 240 are similarly contemplated, including a vector of numbers, bit fields, or any other encoding capable of representing a sequence of DNA.

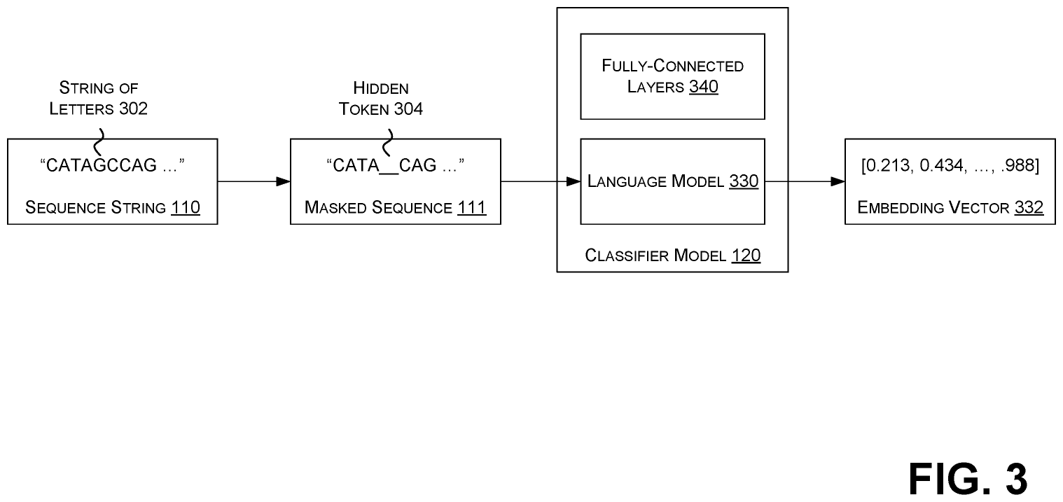

Raw DNA sequences 240 may be used with an unsupervised training algorithm, such as a masked language model (MLM) algorithm, as discussed in detail below in conjunction with FIG. 3. For example, unannotated sequences 240 may be used to pre-train a language model. Since they do not need to be annotated, the number of raw DNA sequences may be large—tens of millions or more.

Training engine 270 may also use taxonomy 250 to train DNA classifier model 120. Taxonomy 250 may be any classification system, such as the hierarchy used by biologists to classify all living organisms. This taxonomy, as discussed above in conjunction with FIG. 1, places a living organism in a 7-level hierarchy. However, hierarchies of any depth are similarly contemplated, as are non-hierarchical taxonomies.

Training engine 270 may also use taxonomy-annotated DNA sequences 260. Each of these sequences has been associated with a location in taxonomy 250. For example, a DNA sequence from a giraffe could be associated with a location in taxonomy 250 with domain Eukaryota, kingdom Animalia, phylum Chordata, class Mammalia, order Artiodactyla, family Giraffidae, genus Giraffa, and species G. camelopardalis. Due to the need to be annotated, taxonomy-annotated DNA sequences 260 are often smaller in number than raw DNA sequences 240, e.g., on the order of tens of thousands of entries. Taxonomy-annotated DNA sequences 260 may be used to train DNA classifier model 120 how to locate a DNA sequence within taxonomy 250.

FIG. 3 illustrates using a language model 330 of DNA classifier model 120 to map a string of characters representing nucleotides of DNA sequence string 110 to an embedding vector 332. The embedding vector 332 may also be referred to as an embedding 332. Embedding vectors represent information that DNA classifier model 120 has learned about a DNA sequence. Embedding vectors can be any size, but high-dimensionality vectors are typical and may have hundreds of elements such as, for example, 768 elements.

Language models are often applied to natural languages, such as English. A model of the English language may be trained with a corpus of English documents, such as Wikipedia® pages. Masked Language Modeling is a technique for training a language model by hiding a specific token—such as one or more words—and asking the model to predict the hidden token. A loss function determines whether or not the model guessed correctly, e.g., by comparing the predicted token to the hidden token. The value returned by the loss function is used during back-propagation to compute gradient changes that update the weights of the model. By repeatedly training the model in this way, the model learns the semantics of English or any other natural or artificial language.

One suitable language model that may be adapted for use with DNA sequence strings 110 is the RoBERTa language model. Y. Liu et al., RoBERTa: A Robustly Optimized BERT Pretraining Approach. arXiv:1907.11692v1 (2019). RoBERTa is a robustly optimized method for pretraining natural language processing (NLP) systems that improves on Bidirectional Encoder Representations from Transformers, or BERT. RoBERTa builds on BERT's language masking strategy. The system learns to predict intentionally hidden sections of strings within otherwise unannotated examples. Bidirectional Encoder Representations from Transformers (BERT) is a transformer-based machine learning technique for natural language processing (NLP) pre-training. BERT is a transformer language model with a variable number of encoder layers and self-attention heads. The architecture is similar to the original transformer implementation in Vaswani et al. (2017).

A transformer is a deep learning model that adopts the mechanism of self-attention, differentially weighting the significance of each part of the input data. Like recurrent neural networks (RNNs), transformers are designed to process sequential input data, such as natural language or DNA sequences. However, unlike RNNs, transformers process the entire input all at once. The attention mechanism provides context for any position in the input sequence. Attention mechanisms let a model draw from the state at any preceding point along the sequence. The attention layer can access all previous states and weight them according to a learned measure of relevance, providing relevant information about far-away tokens.

The input string is parsed into tokens by a byte pair encoding tokenizer, and each token is converted via an embedding into a vector. Then, positional information of the token is added to the embedding. The Transformer model used an encoder-decoder architecture. The encoder consists of encoding layers that process the input iteratively one layer after another, while the decoder consists of decoding layers that do the same thing to the encoded output. The function of each encoder layer is to generate encodings that contain information about which parts of the inputs are relevant to each other. It passes its encodings to the next encoder layer as inputs. Each decoder layer does the opposite, taking all the encodings and using their incorporated contextual information to generate an output sequence. To achieve this, each encoder and decoder layer makes use of an attention mechanism. For each input, attention weighs the relevance of every other input and draws from them to produce the output. Each decoder layer has an additional attention mechanism that draws information from the outputs of previous decoders before the decoder layer draws information from the encodings. Both the encoder and decoder layers have a feed-forward neural network for additional processing of the outputs and contain residual connections and layer normalization steps.

When applying Masked Language Modeling to train a language model with DNA sequences, a hidden token 304 comprising one or more letters 302 of a DNA sequence string 110 is chosen, e.g., ‘A’, ‘C’, ‘GC’, or ‘TTTA’. Hidden tokens 304 are selected at random to be hidden, and the language model 330 is asked to predict what was hidden. A DNA sequence string 110 with a hidden token 304 is a masked sequence 111. Initially, the language model 330 has no information about DNA sequences, and so the initial predictions are random guesses. A loss function compares the output of the language model 330—a predicted token—to the actual token that was hidden. The results of the loss function are used to perform gradient descent while training the model. As with English language models, as the DNA language model is trained it will become better at predicting hidden sequences. If language model 330 becomes effective at predicting hidden tokens, it is likely that embedding vectors 332 generated by the language model 330 will encode meaningful semantics of DNA sequence string 110. Language model 330 may be trained with a large number of sequences, e.g., 50 million sequences, in order to obtain a good understanding of the patterns present in DNA.

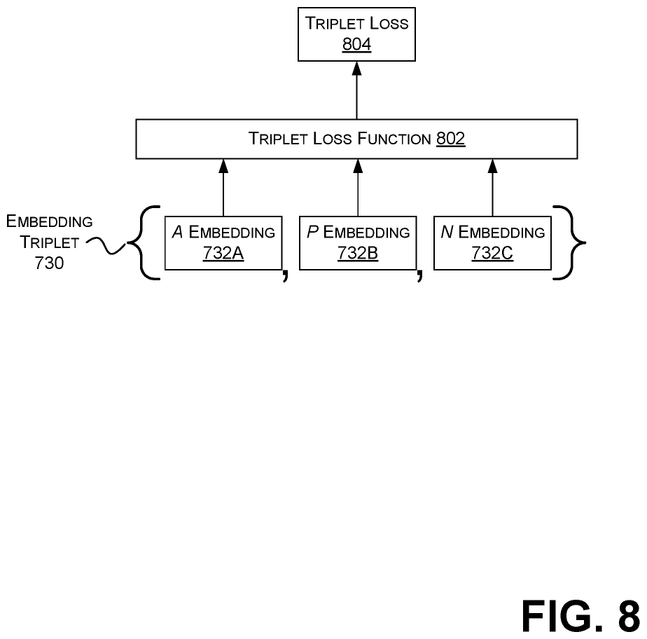

Once language model 330 has been trained it may be used to infer a missing section of DNA. Language model 330 may also be used to generate an embedding 332 for a given DNA sequence string 110. For example, language model 330 may generate a high dimensionality embedding vector 332 that encodes what language model 330 has learned about sequence string 110. These embeddings may be compared to each other to identify patterns in the training data. For example, clusters of embeddings, as measured by Euclidian distance, may be composed of embeddings that are related to each other in some way. As discussed below in conjunction with FIG. 8, by training the model to cluster based on taxonomy similarity, clusters of embeddings will come to reflect co-location in a taxonomy. Using embeddings to identify relationships between DNA sequences is one example of how language model 330 has learned from training data that is raw DNA sequences 240. Instead of merely memorizing information, a well-trained language model 330 learns relationships that exist in the data.

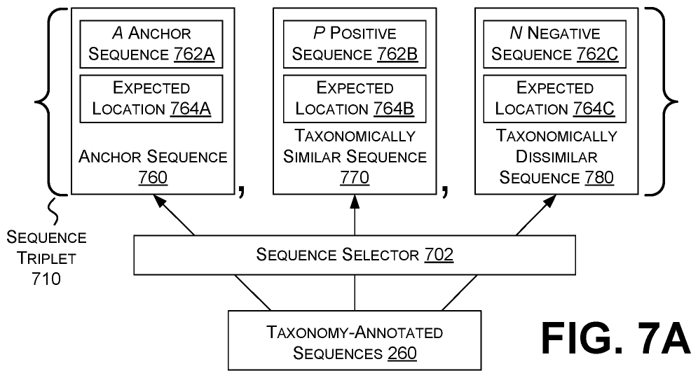

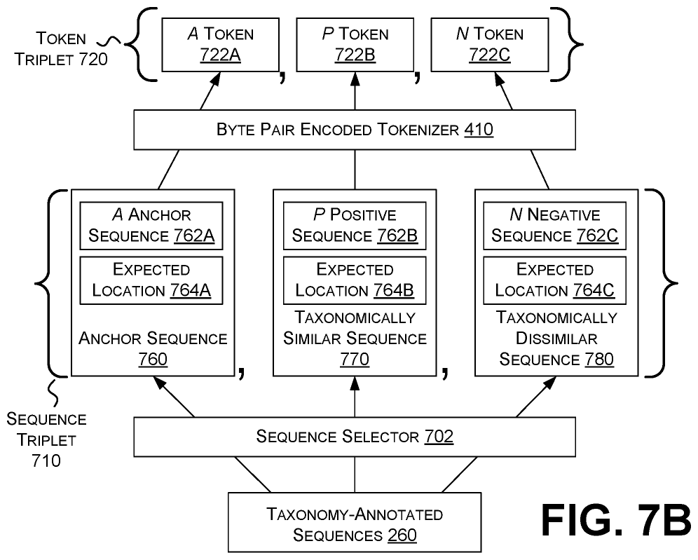

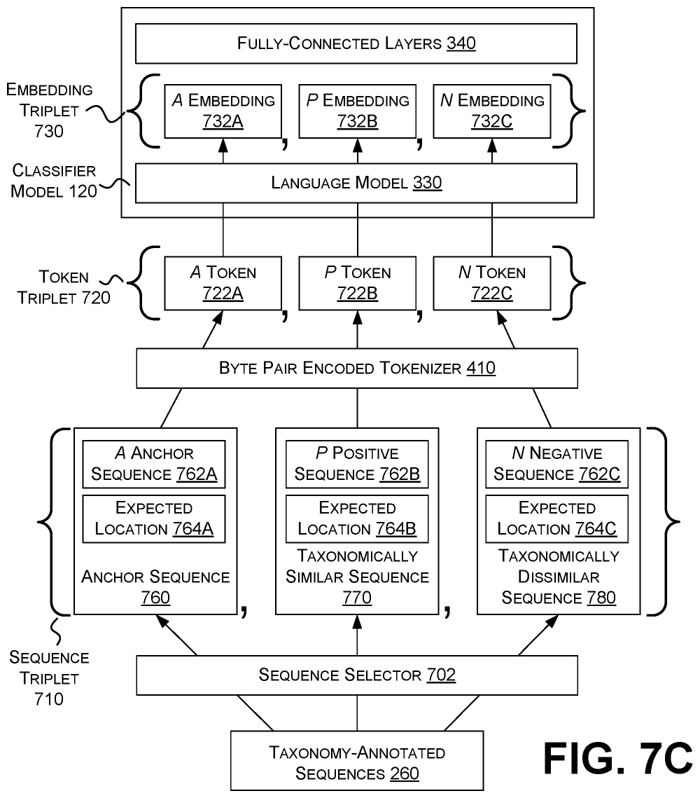

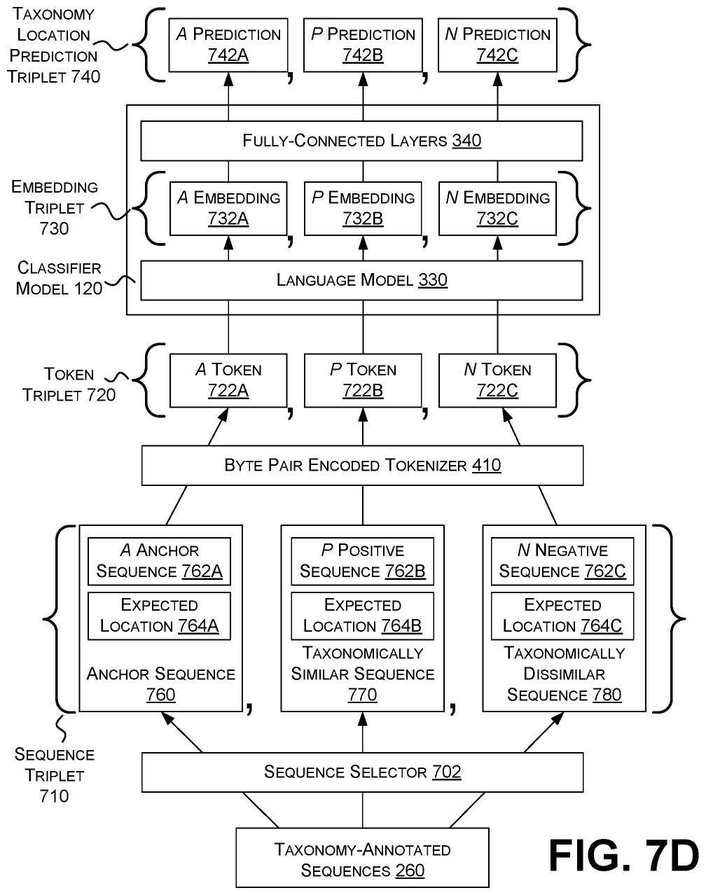

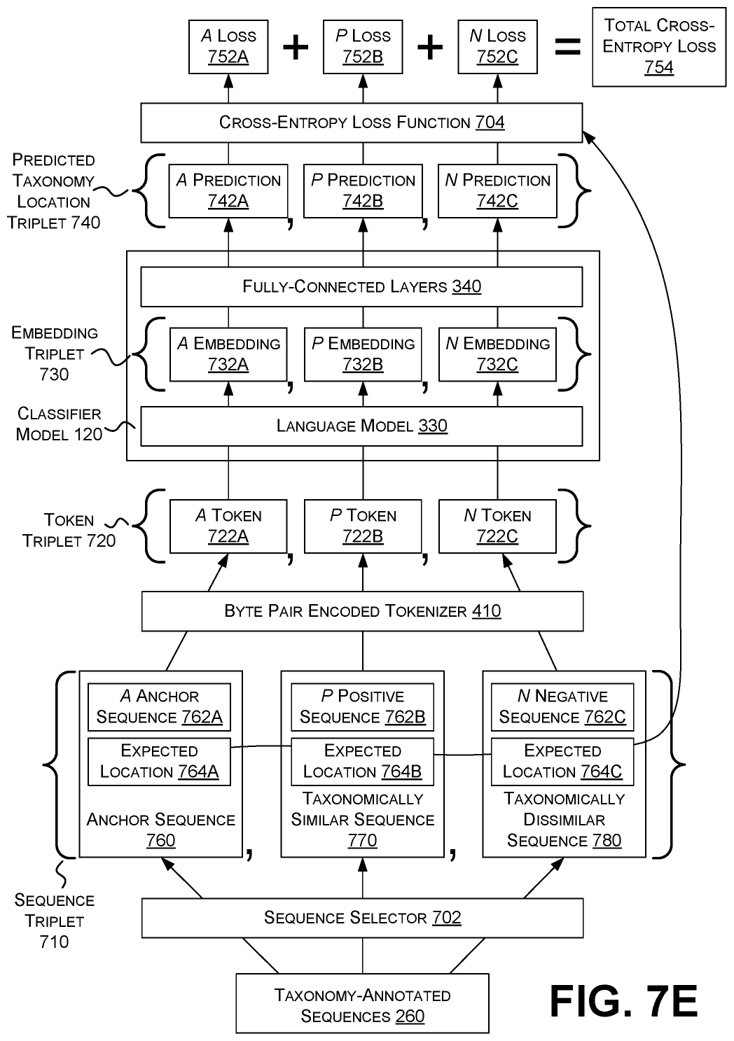

Embeddings 332 generated by language model 330 may also be provided as input to fully-connected layers 340, which are trained to predict a location within a taxonomy. Predicting a location within a taxonomy is discussed in more detail below in conjunction with FIG. 7D.

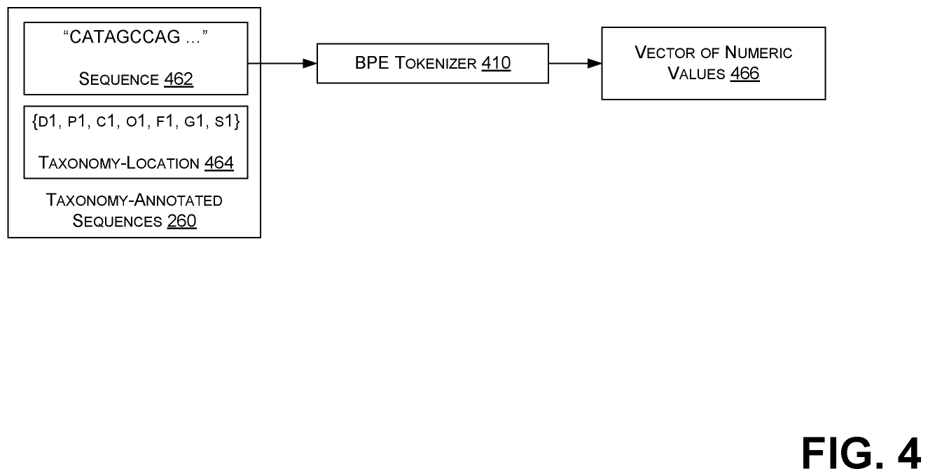

FIG. 4 illustrates converting text-based DNA sequence data 462 into a vector of numeric values 466 that can be provided as input to model 120. In some configurations, DNA sequence data 462 is represented as a string of characters that are first converted to an initial vector of numerical values 466. For example, Byte Pair Encoded (BPE) tokenizer 410 may convert a text-based DNA sequence 462 into a vector of numeric values 466. Vector of numeric values 466 may have the same length as an embedding vector 332 of the pre-trained language model 330.

BPE tokenizer 410 may be a hard-coded mechanism which, for example, translates each ‘A’ into a 0, each ‘C’ into a 1, each G′ into a 2, and each ‘T’ into a 3. However, BPE tokenizer 410 may map pairs of letters, or other patterns of letters, to specific numbers. Other known tokenizing techniques are similarly contemplated.

Each of taxonomy-annotated DNA sequences 260 also include taxonomy location 464. Taxonomy location 464 indicates where sequence 462 appears in taxonomy 250.

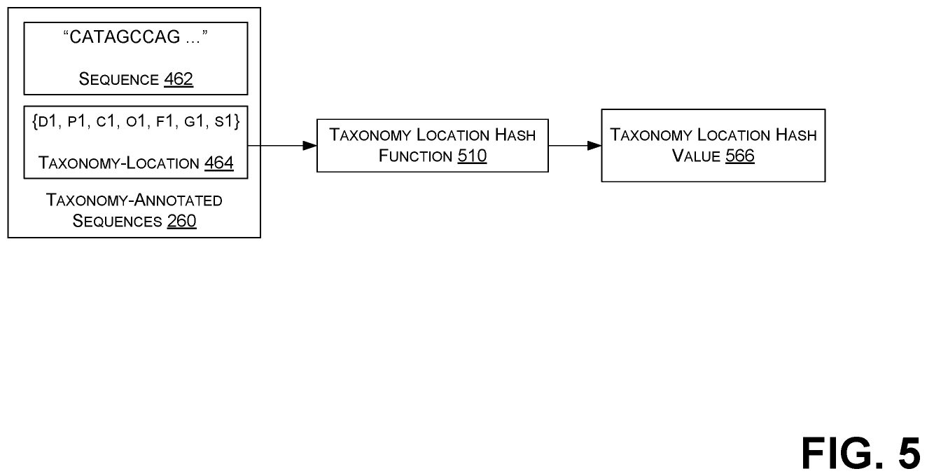

FIG. 5 illustrates converting taxonomy location 464 into a numeric representation—a taxonomy location hash value 566. As referred to herein, a hash is a numeric value generated by a function that maps an input, such as a taxonomy location 464 within taxonomy 250, to a numeric value, such as taxonomy location hash value 566. Mapping locations in a hierarchy to a numeric value is similar to how image classification models indicate how an image was classified with a numerical value. For example, an image classification model may identify whether an image contains a cat or a dog, where images containing a cat are indicated with the value “0” and images containing a dog are indicated with the value “1”.

In some configurations, taxonomy location 464 is converted to a single hash value 566. For example, when the taxonomy 250 is the hierarchy commonly used by biologists for classifying living organisms, each combination of domain, phylum, class, order, family, genus, and species may be mapped to a single taxonomy location hash value 566. For instance, an organism with a taxonomy location 464 of {d1, p1, c1, o1, f1, g1, s1} may be assigned hash h₀ of 0. A mapping of hierarchy locations to taxonomy location hash values 566 may be maintained so that the levels of a hierarchy may be retrieved for a given taxonomy location hash value 566.

In some configurations, another hierarchy, no matter how similar to or different from taxonomy location 464, will be assigned a different hash h₁ that is unrelated to h₀. For example, a taxonomy-location of {d1, p1, c1, o1, f1, g1, s2}, which only differs in the species, may be given the hash h₁ of 42. When hash values are unrelated to one another, they may be chosen at random or by a pseudo-random hashing function, so long as no two locations in the taxonomy 250 are assigned the same hash 566. In other configurations, taxonomy location hash value 566 is not selected at random but is constructed from taxonomy location 464. Hash value 566 may or may not be constructed such that taxonomy locations that are closer together map to taxonomy location hash values 566 that are related in some way, e.g., having the same digit in a particular location.

In another configuration, instead of assigning a single hash value to each location in taxonomy 250, hash value 566 may include more than one number. For example, each identifier of each level of the taxonomy hierarchy may be assigned a number. For example, animalia, plantae, and fungi may be assigned the values 1, 2, and 3, respectfully. The numbers assigned to each identifier may be unique within a particular level, or they may be unique across all levels. Together, these identifiers uniquely identify each location in taxonomy 250. The result is that each taxonomy is represented as a vector of numeric values. Continuing the example of the taxonomy location 464 {d1, p1, c1, o1, f1, g1, s1}, a unique value may be assigned for the domain, phylum, class, etc. The resulting vector of numeric values could be {1, 2, 4, 8, 5, 31, 52}, for example.

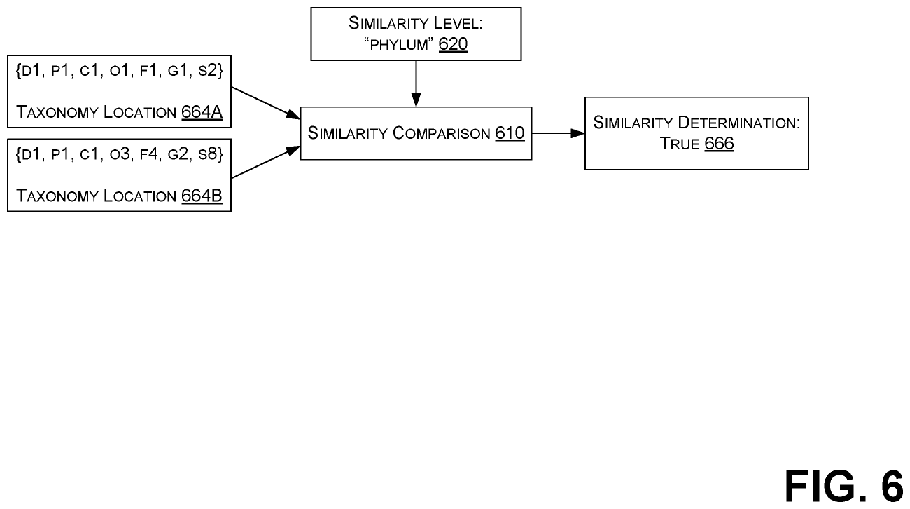

FIG. 6 illustrates similarity comparison engine 610 for determining if taxonomy locations 664 are similar. In some configurations, similarity level 620 is a level in the hierarchy of taxonomy 250. If similarity comparison engine 610 determines that two taxonomy locations 664 are the same from the highest level in the hierarchy down to and including similarity level 620, then similarity determination 666 will be true. If at least one of these levels is not the same then similarity determination 666 for the taxonomy locations 664 will be false.

For example, taxonomy-location 664A is {d1, p1, c1, o1, f1, g1, s2} and taxonomy 664B is {d1, p1, c1, o3, f4, g2, s8}. If similarity level 620 is “phylum”, then similarity comparison engine 610 would determine that these taxonomy locations 664 are similar because the highest level—domain—down to and including the similarity level of “phylum” are the same. Specifically, the domains of both locations are “d1” and the phyla of both locations are “p1”. Locations 664 would also be considered similar for a similarity level of “class”. However, if the similarity level were “order”, then taxonomies 664A and 664B, which have orders “o1” and “o3”, respectively, would be considered dissimilar.

Applying similarity threshold 620 to determine whether two taxonomy locations 664 are similar enables a hybrid training technique that combines similarity with classification. In addition to classification, in which DNA classifier model 120 is trained based on whether an input DNA sequence string 110 yielded the expected taxonomy location 464, model 120 may also be trained on whether input DNA sequence string 110 is similar to another DNA sequence.

FIG. 7A illustrates generating DNA sequence triplets 710 with which to train DNA classifier model 120. Sequence selector 702 constructs DNA sequence triplets 710 such that two of the sequences are similar and two of the sequences are dissimilar. In some configurations, anchor sequence 760 is selected first from taxonomy-annotated DNA sequences 260. Anchor sequence 760 may be selected at random, or anchor sequence 760 may be selected based on inclusion in a particular branch of taxonomy 250, or any other criteria. Then, taxonomically similar DNA sequence 770 is selected from sequences 260 because it is similar to anchor sequence 760, and taxonomically dissimilar DNA sequence 780 is selected from sequences 260 because it is dissimilar to anchor sequence 760.

In some configurations, training engine 270 may systematically generate triplets out of some or all of taxonomy-annotated DNA sequences 260. In some configurations, similarity and dissimilarity are determined by similarity comparison engine 610 discussed above in conjunction with FIG. 6. Generating triplets in this way, and applying a triplet loss function as discussed below in conjunction with FIG. 8, trains DNA classifier model 120 to co-locate within the embedding space sequences that are more closely related by taxonomy 250. Specifically, DNA classifier model 120 learns to represent similar sequences with embedding vectors that are closer, in terms of Euclidian distance, than dissimilar sequences.

Each of sequences 760, 770, and 780 include the actual DNA sequence 762—e.g., strings of letters representing nucleotides, and an expected location 764 within taxonomy 250. Throughout this document the following nomenclature is used to reference to triplets used for training. A is used to refer to a value or computation associated with an Anchor sequence 760. P—for Positive similarity—is used to refer to a value or computation associated with a taxonomically similar sequence 770. N—for Negative similarity—is used to refer to a value or computation associated with a taxonomically dissimilar sequence 780.

In some configurations, sequence selector 702 generates batches of triplets. When training DNA classifier model 120, training engine 270 may process a batch of triplets through model 120, accumulating the values returned by the loss function. Backpropagation may then be performed with the accumulated loss function value.

FIG. 7B illustrates BPE tokenizer 410 processing A anchor sequence 762A, P positive sequence 762B, and N negative sequence 762C into A token 722A, P token 722B, and N token 722C, respectively, as discussed above in conjunction with FIG. 4. In some configurations, each of the DNA sequences 762 are processed separately to create tokens 722 of token triplet 720. Tokens 722 may be vectors with the same number of elements as embedding vectors 332, although any other number of elements, or any other type of encoding, are similarly contemplated, so long as language model 330 is configured to accept tokens 722 as inputs.

FIG. 7C illustrates language model 330 inferring embedding vectors 732 from tokens 722. Each token 722 of tokens triplet 720 is provided one at a time as input to pre-trained language model 330 of DNA classifier model 120. The resulting A embedding 732A, P embedding 732B, and N embedding 732C of respective tokens 722A, 722B, and 722C may be used in two different ways. The first, as discussed below in conjunction with FIGS. 7D-7E, provides embedding vectors 732 as inputs to fully-connected layers 340 of DNA classifier model 120, the results of which are provided to cross-entropy loss functions. The second, as discussed below in conjunction with FIG. 8, provides embedding triplet 730 to a triplet loss function.

FIG. 7D illustrates providing the embedding vectors 732 to fully-connected layers 340, which yields a taxonomy location prediction triplet 740 of taxonomy location predictions 742. As with language model 330 and BPE tokenizer 410, each of embedding vectors 732 are provided to fully-connected layers 340 one at a time to generate individual taxonomy location predictions 742. In some configurations, predictions 742 are valid taxonomy location hash values 566.

In some configurations, the fully connected layers 340 of DNA classifier model 120 includes a Softmax layer. The Softmax layer generates a probability distribution indicating a probability associated with various taxonomy locations that could be associated with the input DNA sequence string 110. Typically, the taxonomy location with the highest probability is selected as the prediction 742.

FIG. 7E illustrates providing taxonomy location predictions 742, along with the corresponding expected locations 764 in the taxonomy 250, to cross-entropy loss function 704. In many forms of machine learning, models are trained using a loss function. Specifically, during training, annotated training data is provided as input to the model, and the resulting model output is tested against an expected output contained in the annotated training data. The difference between the actual output and the expected output is referred to as loss, which is used to adjust the model's weights. Specifically, annotated training data includes model inputs and a corresponding model output that, by definition, is correct. The loss function compares the output of the model to the correct value. The result of the loss function is then used to adjust the weights of the model, e.g., via back-propagation.

One common loss function is a cross-entropy loss function. Cross-entropy loss functions encourage models to predict the correct output. In general, the more that the model is correct, the smaller the value returned by the loss function. If the model outputs the exact correct output, then the loss function will return zero. The inverse is also true: the further the model is from the correct output, the larger the value returned by the loss function. When a model output is either correct or incorrect, then a binary cross-entropy loss function may be used.

In some configurations, cross-entropy loss function 704 is a binary cross-entropy loss function that determines whether a taxonomy location prediction 742 matches the expected location 764 in the taxonomy 250. For example, predicted taxonomy location 742A and corresponding expected taxonomy location 764A may be processed by cross-entropy loss function 704 to generate Anchor loss 752A. In some configurations, the loss values 752 associated with each prediction 742 is added together to yield total cross-entropy loss 754.

While binary cross-entropy loss functions are used as examples throughout this document, other types of cross-entropy loss functions are similarly contemplated, as are other types of loss functions.

FIG. 8 illustrates applying a triplet loss function 802 to embedding vectors 732. Triplet loss function 802 determines a triplet loss 804 which is used to encourage DNA classifier model 120 to produce embeddings such that, if two DNA sequence strings 110 are similar, their embeddings 332 are close to each other in the embedding space. Triplet loss function 802 also encourages the DNA classifier model 120 to produce embeddings that, if the DNA sequences are dissimilar, their embedding vectors are far away from each other in the embedding space.

In this context, two embeddings may be considered close if the distance between them is less than the distances between a defined percentage of pairs of taxonomy-annotated embedding vectors 260. Embeddings may also be considered close if the distance between them is less than a threshold absolute distance. Embedding vectors may be considered far from each other if the distance between them is greater than the distances between a defined percentage of pairs of taxonomy-annotated embedding vectors 260. As referred to herein, embedding space is an N-dimensional space occupied by N-element embedding vectors.

One example of a triplet loss function L is defined as:

L(a,p,n)=max{d(aᵢ,pᵢ)−d(aᵢ,nᵢ)+margin,0}

Where a, p, and n are embedding vectors of DNA sequences. Specifically, a is an anchor—i.e., a is the embedding vector of a DNA sequence selected from taxonomy-annotated DNA sequences 260. p stands for positive and refers to a sequence that is similar to the anchor, where similarity refers to having at least a threshold number of common ancestors beginning at the root of the taxonomy, as defined above in conjunction with FIG. 6. n stands for negative and refers to a sequence that is not similar to the anchor. “d(x, y)” refers to the Euclidian distance between x and y.

Loss function L returns 0 when the positive sequence p is sufficiently close to the anchor a and the negative sequence n is sufficiently far away from anchor a. Whether p is sufficiently close and n is sufficiently far away is determined by the value of the margin. The value returned by L decreases as the Euclidian distance between a and p decreases and as the distance between a and n increases.

L takes the max of two terms, “d(aᵢ, pᵢ)−d(aᵢ, nᵢ)+margin” and “0” such that if “d(aᵢ, pᵢ)−d(aᵢ,nᵢ)+margin” is negative, max will return “0”. Zero means that the model's prediction is correct and does not need to learn anything from this annotated input. However, if “d(aᵢ, pᵢ)−d(aᵢ,nᵢ)+margin” is greater than “0”, then L will return this non-zero number, and training engine 270 will use this number to adjust the weights of DNA classifier model 120 during back propagation.

In general, L tends to return a value closer to zero for smaller values of d(aᵢ, pᵢ) and larger values of d(aᵢ,nᵢ). In other words, as the positive embedding 732B moves closer to the embedding of the anchor sequence 732A and as the negative embedding 732C moves further from the embedding of the anchor sequence 732A, L tends towards zero.

Loss function L returns zero when the distance between the negative embedding 732C and the anchor embedding 732A exceeds the distance between the positive embedding 732B and the anchor embedding 732A by at least the margin. For example, if d(aᵢ, pᵢ) is 1.8, d(aᵢ,nᵢ) is 3.3, and margin is 1, then L=max(1.8−3.3+1, 0}=max{−0.5, 0}=0.

L returns a non-zero number when the negative sequence is closer to the anchor than the positive sequence. For example, if d(aᵢ, pᵢ) is 1.8, d(aᵢ, nᵢ) is 1.3, and margin is 1, then L=max(1.8−1.3+1, 0}=max{1.5, 0}=1.5. L also returns a non-zero number when the distance between the negative sequence and the anchor is greater than the distance between the positive sequence and the anchor by less than the margin. For example, if d(aᵢ, pᵢ) is 1.8, d(aᵢ, nᵢ) is 2.3, and margin is 1, then L=max(1.8−2.3+1, 0}=max{0.5, 0}=0.5.

Margin controls how aggressively the model should be trained. If margin is 0, then the model is considered to have predicted correctly so long as the positive sequence is at least as close to the anchor as the negative sequence. This means that the positive and negative sequences could be equally distant from the anchor and still be considered correct. As margin is increased, in order to L to continue to return zero, the positive sequence must be increasingly closer to the anchor than the negative sequence. Using a large margin value is like telling the model it has to be even more sure before two sequences can be considered similar. Typical values for margin are 1 or 2, but different values will have better or worse results for different datasets, and so any value is similarly contemplated.

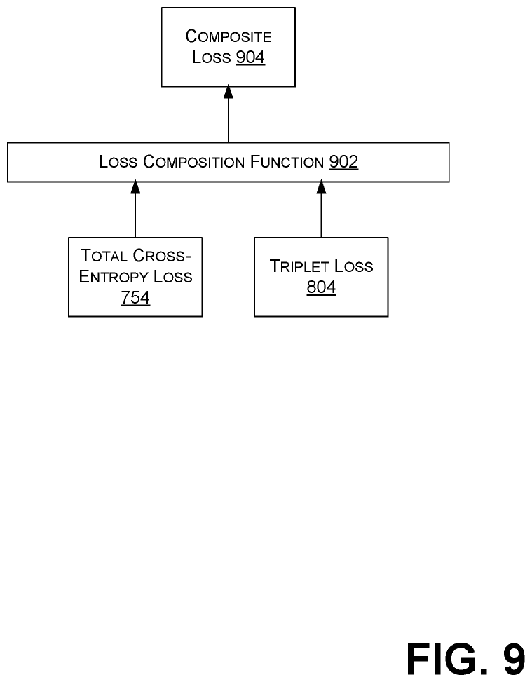

FIG. 9 illustrates combining the results of cross-entropy loss functions and triplet loss functions into a composite loss 904. In some configurations, loss composition function 902 combines the results of two loss functions—total cross-entropy loss 754 and triplet loss 804—by adding them together. The total cross-entropy loss 754 component of composite loss 904 trains DNA classifier model 120 to correctly predict the taxonomy for a given sequence. The triplet loss 804 component trains DNA classifier model 120 such that DNA sequence strings 110 that are closer together.

The value returned by loss composition function 902 may be used to train model 120, e.g., via backpropagation. Backpropagation may be applied to the fully-connected layers 340. This has the effect of training model 120 to correctly classify DNA sequences within taxonomy 250. Backpropagation may then continue through pre-trained language model 330. This has the effect of adjusting embedding vector representations of DNA sequences to cluster based on similarity.

In some configurations, when model 120 is applied to a test dataset, accuracies of up to 0.736 have been observed after a single epoch of training—i.e., upwards of 74% of DNA sequences are accurately classified into the correct location within a taxonomy. This is an unexpectedly favorable result given that a random rate of success would be the inverse of the number of organisms in taxonomy-annotated DNA sequences 260. For example, if there 100 organisms annotated in the taxonomy-annotated DNA sequences 260, the expected rate of correctly classifying a DNA sequence by random would be 1/100=0.01.

As a test of how close positive DNA sequences are from an anchor and how far apart negative DNA sequences are from an anchor after one epoch: an average positive pair distance of 3.22 and an average negative pair distance of 5.42 were observed. This shows that sequences that are similar to each other, as defined above in conjunction with FIG. 6, are closer in the embedding space than sequences that are dissimilar.

These results are competitive with existing techniques for classifying DNA sequences but are achievable without the extensive human knowledge of biology that are used in these other tools. Advantages of the disclosed techniques include the ability to train model 120 for particular datasets that may outperform the generic algorithms of existing tools. The disclosed techniques also provide an advantage in that they enable visualization of DNA sequence relatedness, as discussed below in conjunction with FIGS. 10A and 10B.

FIG. 10A illustrates a 2D plot of embedding vectors shaded based on the domain they have been classified as being a part of. Each circle represents an embedding vector 732 of a DNA sequence. Dimensionality reduction has been used to generate this 2D depiction from higher dimensional embedding vectors. For example, an embedding vector with 768 dimensions may be reduced to a 2D representation. As illustrated, cluster 1010 represents a cluster of DNA sequences that are associated with the archaea domain. Similarly, bacteria cluster 1020 represents a cluster of DNA sequences that are associated with the bacteria domain. Existing, procedural techniques for DNA sequence classification are not able to generate these types of associations.

FIG. 10B illustrates a 2D depiction of embedding vectors shaded by phylum. The circles in FIG. 10B are unchanged from FIG. 10A—only the shading has changed to reflect grouping by phylum. Similar to FIG. 10A discussed above, phylum clusters 1030 further illustrate clusters of DNA sequences.

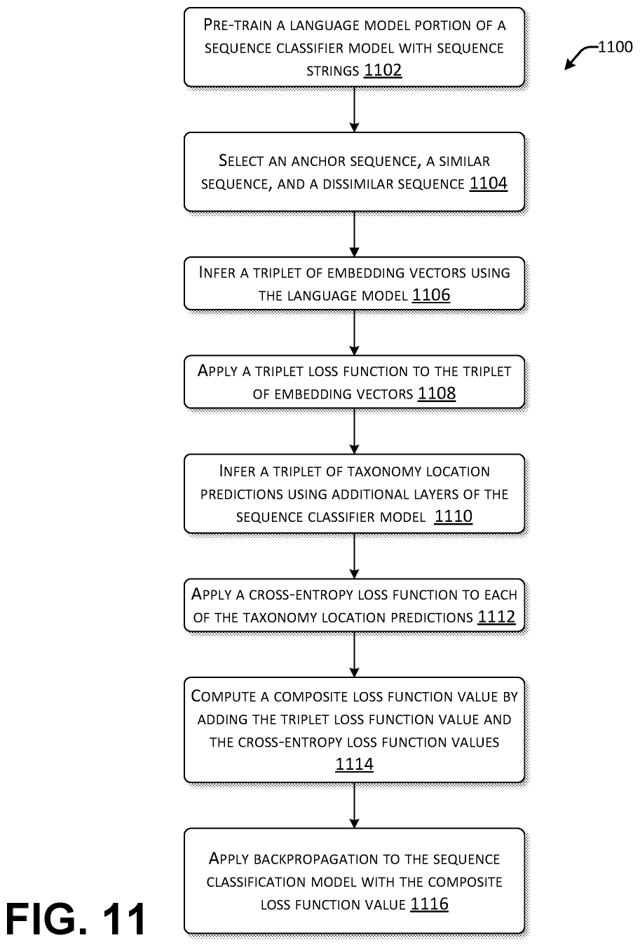

Turning now to FIG. 11, aspects of a routine for DNA taxonomy classification using language models and triplet loss is shown and described. For ease of understanding, the processes discussed in this disclosure are delineated as separate operations represented as independent blocks. However, these separately delineated operations should not be construed as necessarily order dependent in their performance. The order in which the process is described is not intended to be construed as a limitation, and any number of the described process blocks may be combined in any order to implement the process or an alternate process. Moreover, it is also possible that one or more of the provided operations is modified or omitted.

With reference to FIG. 11, routine 1100 begins at operation 1102, where language model 330 is pre-trained to understand DNA sequences using a masked language model technique.

Next at operation 1104, a DNA sequence triplet 710 is created by selecting an anchor DNA sequence 760 from taxonomy-annotated DNA sequences 260. Then, a taxonomically similar DNA sequence 770 and a taxonomically dissimilar DNA sequence 780 are selected to fill out the DNA sequence triplet 710.

Next at operation 1106, language model 330 is used to infer an embedding triple 730 with one embedding vector 732 for each of the DNA sequences in DNA sequence triplet 710.

Next at operation 1108, training engine 270 applies a triplet loss function 802 to the embedding vector triplet 720, yielding a triplet loss 804.

Next at operation 1110, a triplet 730 of embedding vectors 732 are inferred from fully-connected layers 340 of DNA classifier model 120. In some configurations, layers 340 are partially connected.

Next at operation 1112, training engine 270 applies a cross-entropy loss function 704 to each of the embedding vectors 732 and the corresponding expected locations 764 in the taxonomy 250. This yields loss values 752 for each of the anchor, positive, and negative DNA sequences.

Next at operation 1114, a composite loss 904 is computed by adding the triplet loss 804 and the total cross-entropy loss 754 summed from the individual cross-entropy loss values 752.

Routine 1100 then proceeds to operation 1116, where backpropagation is applied to DNA classifier model 120 with composite loss 904.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, in special-purpose digital logic, and any combination thereof. It should be appreciated that more or fewer operations can be performed than shown in the figures and described herein. These operations can also be performed in a different order than those described herein.

It also should be understood that the illustrated methods can end at any time and need not be performed in their entireties. Some or all operations of the methods, and/or substantially equivalent operations, can be performed by execution of computer-readable instructions included on a computer-storage media, as defined below. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

For example, the operations of the routine 1100 are described herein as being implemented, at least in part, by modules running the features disclosed herein can be a dynamically linked library (DLL), a statically linked library, functionality produced by an application programing interface (API), a compiled program, an interpreted program, a script or any other executable set of instructions. Data can be stored in a data structure in one or more memory components. Data can be retrieved from the data structure by addressing links or references to the data structure.

Although the following illustration refers to the components of the figures, it should be appreciated that the operations of the routine 1100 may be also implemented in many other ways. For example, the routine 1100 may be implemented, at least in part, by a processor of another remote computer or a local circuit. In addition, one or more of the operations of the routine 1100 may alternatively or additionally be implemented, at least in part, by a chipset working alone or in conjunction with other software modules. In the example described below, one or more modules of a computing system can receive and/or process the data disclosed herein. Any service, circuit or application suitable for providing the techniques disclosed herein can be used in operations described herein.

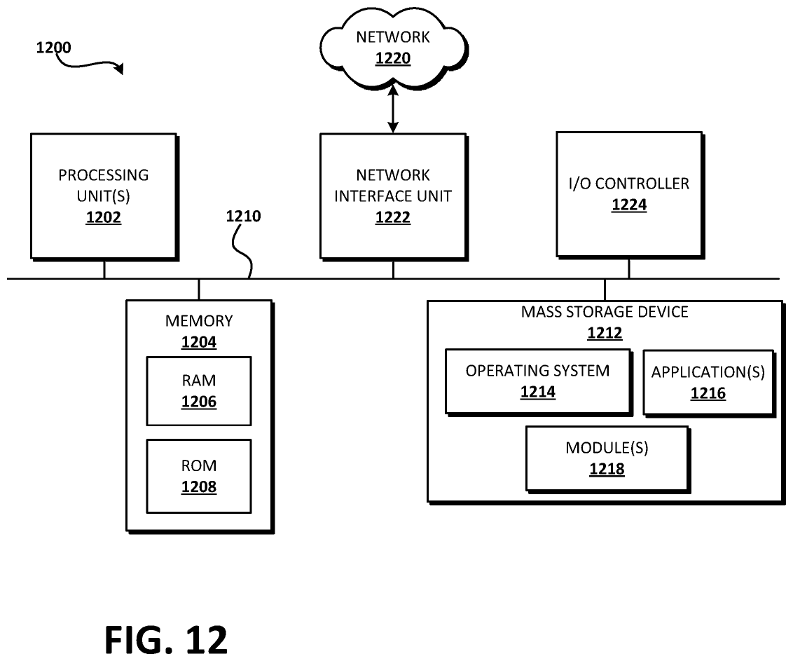

FIG. 12 shows additional details of an example computer architecture 1200 for a device, such as a computer or a server configured as part of the systems described herein, capable of executing computer instructions (e.g., a module or a program component described herein). The computer architecture 1200 illustrated in FIG. 12 includes processing unit(s) 1202, a memory 1204, including a random-access memory 1206 (“RAM”) and a read-only memory (“ROM”) 1208, and a system bus 1210 that couples the memory 1204 to the processing unit(s) 1202.

Processing unit(s), such as processing unit(s) 1202, can represent, for example, a CPU-type processing unit, a GPU-type processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), etc.

A basic input/output system containing the basic routines that help to transfer information between elements within the computer architecture 1200, such as during startup, is stored in the ROM 1208. The computer architecture 1200 further includes a mass storage device 1212 for storing an operating system 1214, application(s) 1216, modules 1218, and other data described herein.

The mass storage device 1212 is connected to processing unit(s) 1202 through a mass storage controller connected to the system bus 1210. The mass storage device 1212 and its associated computer-readable media provide non-volatile storage for the computer architecture 1200. Although the description of computer-readable media contained herein refers to a mass storage device, it should be appreciated by those skilled in the art that computer-readable media can be any available computer-readable storage media or communication media that can be accessed by the computer architecture 1200.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random access memory (RAM), static random-access memory (SRAM), dynamic random-access memory (DRAM), phase change memory (PCM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 1200 may operate in a networked environment using logical connections to remote computers through the network 1220. The computer architecture 1200 may connect to the network 1220 through a network interface unit 1222 connected to the system bus 1210. The computer architecture 1200 also may include an input/output controller 1224 for receiving and processing input from a number of other devices, including a keyboard, mouse, touch, or electronic stylus or pen. Similarly, the input/output controller 1224 may provide output to a display screen, a printer, or other type of output device.

It should be appreciated that the software components described herein may, when loaded into the processing unit(s) 1202 and executed, transform the processing unit(s) 1202 and the overall computer architecture 1200 from a general-purpose computing system into a special-purpose computing system customized to facilitate the functionality presented herein. The processing unit(s) 1202 may be constructed from any number of transistors or other discrete circuit elements, which may individually or collectively assume any number of states. More specifically, the processing unit(s) 1202 may operate as a finite-state machine, in response to executable instructions contained within the software modules disclosed herein. These computer-executable instructions may transform the processing unit(s) 1202 by specifying how the processing unit(s) 1202 transition between states, thereby transforming the transistors or other discrete hardware elements constituting the processing unit(s) 1202.

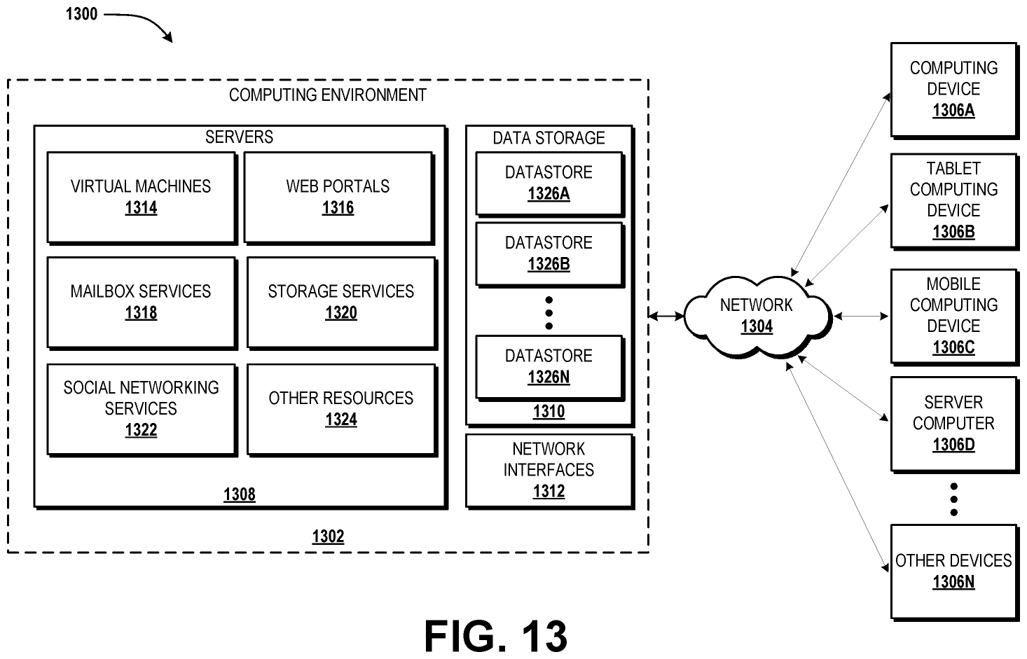

FIG. 13 depicts an illustrative distributed computing environment 1300 capable of executing the software components described herein. Thus, the distributed computing environment 1300 illustrated in FIG. 13 can be utilized to execute any aspects of the software components presented herein. For example, the distributed computing environment 1300 can be utilized to execute aspects of the software components described herein.

Accordingly, the distributed computing environment 1300 can include a computing environment 1302 operating on, in communication with, or as part of the network 1304. The network 1304 can include various access networks. One or more client devices 1306A-606N (hereinafter referred to collectively and/or generically as “clients 1306” and also referred to herein as computing devices 1306) can communicate with the computing environment 1302 via the network 1304. In one illustrated configuration, the clients 1306 include a computing device 1306A such as a laptop computer, a desktop computer, or other computing device; a slate or tablet computing device (“tablet computing device”) 1306B; a mobile computing device 1306C such as a mobile telephone, a smart phone, or other mobile computing device; a server computer 1306D; and/or other devices 1306N. It should be understood that any number of clients 1306 can communicate with the computing environment 1302.

In various examples, the computing environment 1302 includes servers 1308, data storage 1310, and one or more network interfaces 1312. The servers 1308 can host various services, virtual machines, portals, and/or other resources. In the illustrated configuration, the servers 1308 host virtual machines 1314, Web portals 1316, mailbox services 1318, storage services 1320, and/or, social networking services 1322. As shown in FIG. 13 the servers 1308 also can host other services, applications, portals, and/or other resources (“other resources”) 1324.

As mentioned above, the computing environment 1302 can include the data storage 1310. According to various implementations, the functionality of the data storage 1310 is provided by one or more databases operating on, or in communication with, the network 1304. The functionality of the data storage 1310 also can be provided by one or more servers configured to host data for the computing environment 1302. The data storage 1310 can include, host, or provide one or more real or virtual datastores 1326A-626N (hereinafter referred to collectively and/or generically as “datastores 1326”). The datastores 1326 are configured to host data used or created by the servers 1308 and/or other data. That is, the datastores 1326 also can host or store web page documents, word documents, presentation documents, data structures, algorithms for execution by a recommendation engine, and/or other data utilized by any application program. Aspects of the datastores 1326 may be associated with a service for storing files.

The computing environment 1302 can communicate with, or be accessed by, the network interfaces 1312. The network interfaces 1312 can include various types of network hardware and software for supporting communications between two or more computing devices including, but not limited to, the computing devices and the servers. It should be appreciated that the network interfaces 1312 also may be utilized to connect to other types of networks and/or computer systems.

It should be understood that the distributed computing environment 1300 described herein can provide any aspects of the software elements described herein with any number of virtual computing resources and/or other distributed computing functionality that can be configured to execute any aspects of the software components disclosed herein. According to various implementations of the concepts and technologies disclosed herein, the distributed computing environment 1300 provides the software functionality described herein as a service to the computing devices. It should be understood that the computing devices can include real or virtual machines including, but not limited to, server computers, web servers, personal computers, mobile computing devices, smart phones, and/or other devices. As such, various configurations of the concepts and technologies disclosed herein enable any device configured to access the distributed computing environment 1300 to utilize the functionality described herein for providing the techniques disclosed herein, among other aspects.

### CONCLUSION

The present disclosure is supplemented by the following example clauses.

Clause 1. A method comprising: generating a masked sequence (111) by masking one or more letters (302) of a (DNA) sequence string (110); training a language model (330) of a (DNA) classifier model (120) with the masked sequence (111); selecting an anchor sequence (760) from a plurality of taxonomy-annotated (DNA) sequences (260), wherein each of the plurality of taxonomy-annotated sequences (260) is annotated with an expected taxonomy location (464) within a taxonomy (250); selecting a taxonomically similar (DNA) sequence (770) that shares a defined number of levels of the taxonomy (250) with the anchor (DNA) sequence (760); selecting a taxonomically dissimilar (DNA) sequence (780) that does not share the defined number of levels of the taxonomy (250) with the anchor sequence (760); inferring an embedding vector (732) from the language model (330) for each of the anchor sequence (760), the taxonomically similar (DNA) sequence (770), and the taxonomically dissimilar (DNA) sequence (780); computing a triplet loss (804) by evaluating a triplet loss function (802) with the embedding vectors (732); inferring a taxonomy location prediction (742) from fully-connected layers (340) of the classifier model (120) for each of the embedding vectors (732); converting each expected taxonomy location (464) of the anchor (DNA) sequence (760), the taxonomically similar sequence (770), and the taxonomically dissimilar (DNA) sequence (780) to taxonomy location hash value (566); generating a total cross-entropy loss (754) by adding the results of invoking a cross-entropy loss function (704) on each of the taxonomy location predictions (742) and corresponding taxonomy location hash values (566); and training the classifier model (120) based on the total cross-entropy loss (754) and the triplet loss (804).

Clause 2. The method of clause 1, wherein the sequence string comprises a first sequence string, further comprising: inferring a location within the taxonomy of a second sequence string by providing the second (DNA) sequence string as input to the classifier model.

Clause 3. The method of clause 1 or 2, wherein the sequence string is encoded as a string of letters (e.g., representing nucleotides), further including: converting the sequence string to one or more numeric values using a byte paired encoding operation.

Clause 4. The method of any of clauses 1 to 3, wherein the taxonomy comprises a hierarchy of classification levels.

Clause 5. The method of clause 4, wherein each expected taxonomy location is comprised of a plurality of identifiers of classification levels within the hierarchy of classification levels.

Clause 6. The method of any of clauses 1 to 5, wherein the machine learning model includes a SoftMax layer that outputs probabilities associated with locations within the taxonomy, and wherein a taxonomy location prediction is inferred for an embedding vector by selecting a highest probability location within the taxonomy from the corresponding SoftMax layer.

Clause 7. The method of any of clauses 1 to 6, wherein each expected taxonomy location is converted to a single number.

Clause 8. The method of any of clauses 1 to 7, wherein the classifier model is trained by applying backpropagation to the classifier model with a composite loss value, wherein the composite loss value is computed by adding the total cross-entropy loss and the triplet loss.

Clause 9. A computing device comprising: one or more processors; a memory in communication with the one or more processors, the memory having computer-readable instructions stored thereupon which, when executed by the one or more processors, cause the computing device to: train a language model (330) of a classifier model (120) with a (DNA) sequence string (110); select an anchor sequence (760) from a plurality of taxonomy-annotated sequences (260), wherein each of the plurality of taxonomy-annotated sequences (260) is annotated with an expected taxonomy location (464) within a taxonomy (250); select taxonomically similar sequence (770) that shares a defined number of levels of the taxonomy (250) with the anchor sequence (760); select a taxonomically dissimilar (DNA) sequence (780) that does not share the defined number of levels of the taxonomy (250) with the anchor sequence (760); infer an embedding vector (732) from the language model (330) for each of the anchor sequence (760), the taxonomically similar sequence (770), and the taxonomically dissimilar (DNA) sequence (780); compute a triplet loss (804) by evaluating a triplet loss function (802) with the embedding vectors (732); infer a taxonomy location prediction (742) from additional layers (340) of the classifier model (120) for each of the embedding vectors (732); converting each expected taxonomy location (464) of the anchor sequence (760), the taxonomically similar sequence (770), and the taxonomically dissimilar (DNA) sequence (780) to taxonomy location hash value (566); generating a total cross-entropy loss (754) by adding the results of invoking a cross-entropy loss function (704) on each of the taxonomy location predictions (742) and corresponding taxonomy location hash value (566); and training the classifier model (120) based on the total cross-entropy loss (754) and the triplet loss (804).

Clause 10. The computing device of clause 9, wherein the sequence string comprises a first sequence string, wherein the instructions further cause the processor to: infer a location within the taxonomy of a second (DNA) sequence string by providing the second sequence string as input to the classifier model.

Clause 11. The computing device of clause 9 or 10, wherein the sequence string is encoded as a string of letters, wherein the instructions further cause the processor to: convert the sequence string to one or more numeric values using a byte paired encoding operation.

Clause 12. The computing device of any of clauses 9 to 11, wherein the taxonomy comprises a hierarchy of classification levels.

Clause 13. The computing device of any of clauses 9 to 12, wherein each expected taxonomy location is converted to a single number, and wherein each of the inferred taxonomy location predictions is a single number.

Clause 14. A method of performing metagenomic analysis on an environmental sample, the method comprising: collecting the environmental sample, the environmental sample comprising genomic material from a plurality of organisms; generating sequence data from the genomic material, wherein the sequence data is not correlated with taxonomic categories of the plurality of organisms; providing the sequence data to a machine learning model, the machine learning model trained with masking language modeling on a corpus of polynucleotide sequences and trained with triplet loss on a corpus of labeled polynucleotide sequences; and receiving from the machine learning model a plurality of taxonomic classifications for the plurality of organisms.

Clause 15. The method of clause 14, wherein the sequence data comprises at least 1,000,000 unique reads.

Clause 16. The method of clause 14 or 15, wherein the environmental sample is collected from a body of water, soil, or the digestive tract of a vertebrate.

Clause 17. The method of any of clauses 14 to 16, wherein the sequence data comprises sequences of 16S ribosomal RNA genes.

Clause 18. The method of any of clauses 14 to 17, wherein the plurality of taxonomic classifications comprises at least one of, domain, kingdom, phylum, class, order, family, genus, or species.

Clause 19. The method of any of clauses 14 to 18, wherein the machine learning model comprises an attention-based bi-directional transformer layer.

Clause 20. The method of any of clauses 14 to 19, wherein the machine learning model is trained using a total loss function that sums cross-entropy loss and triplet loss.

Clause 21. The method of any of clauses 14 to 21, further comprising: generating two-dimensional representations of higher-dimensionality vectors representing the taxonomic classifications for the plurality of organisms; and displaying the two-dimensional representations on a display device.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element.

In closing, although the various techniques have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

What is claimed is:

1. A method comprising:
generating a masked sequence (111) by masking one or more letters (302) of a sequence string (110);
training a language model (330) of a classifier model (120) with the masked sequence (111);
selecting an anchor sequence (760) from a plurality of taxonomy-annotated sequences (260), wherein each of the plurality of taxonomy-annotated sequences (260) is annotated with an expected taxonomy location (464) within a taxonomy (250);
selecting a taxonomically similar sequence (770) that shares a defined number of levels of the taxonomy (250) with the anchor sequence (760);
selecting a taxonomically dissimilar sequence (780) that does not share the defined number of levels of the taxonomy (250) with the anchor sequence (760);
inferring an embedding vector (732) from the language model (330) for each of the anchor sequence (760), the taxonomically similar sequence (770), and the taxonomically dissimilar sequence (780);
computing a triplet loss (804) by evaluating a triplet loss function (802) with the embedding vectors (732);
inferring a taxonomy location prediction (742) from additional layers (340) of the classifier model (120) for each of the embedding vectors (732);
converting each expected taxonomy location (464) of the anchor sequence (760), the taxonomically similar sequence (770), and the taxonomically dissimilar sequence (780) to taxonomy location hash value (566);
generating a total cross-entropy loss (754) by adding the results of invoking a cross-entropy loss function (704) on each of the taxonomy location predictions (742) and corresponding taxonomy location hash value (566); and
training the classifier model (120) based on the total cross-entropy loss (754) and the triplet loss (804).

2. The method of claim 1, wherein the sequence string comprises a first sequence string, further comprising:
inferring a location within the taxonomy of a second sequence string by providing the second sequence string as input to the classifier model.

3. The method of claim 1, wherein the sequence string is encoded as a string of letters, further including:
converting the sequence string to one or more numeric values using a byte paired encoding operation.

4. The method of claim 1, wherein the taxonomy comprises a hierarchy of classification levels.

5. The method of claim 4, wherein each expected taxonomy location is comprised of a plurality of identifiers of classification levels within the hierarchy of classification levels.

6. The method of claim 1, wherein the additional layers of the classifier model include a SoftMax layer that outputs probabilities associated with locations within the taxonomy, and wherein a taxonomy location prediction is inferred for an embedding vector by selecting a highest probability location within the taxonomy from the SoftMax layer.

7. The method of claim 1, wherein each expected taxonomy location is converted to a single number.

8. The method of claim 1, wherein the classifier model is trained by applying backpropagation to the classifier model with a composite loss value, wherein the composite loss value is computed by adding the total cross-entropy loss and the triplet loss.

9. A computing device comprising:
one or more processors;
a memory in communication with the one or more processors, the memory having computer-readable instructions stored thereupon which, when executed by the one or more processors, cause the computing device to:
train a language model (330) of a classifier model (120) with a sequence string (110);
select an anchor sequence (760) from a plurality of taxonomy-annotated sequences (260), wherein each of the plurality of taxonomy-annotated sequences (260) is annotated with an expected taxonomy location (464) within a taxonomy (250);
select taxonomically similar sequence (770) that shares a defined number of levels of the taxonomy (250) with the anchor sequence (760);
select a taxonomically dissimilar sequence (780) that does not share the defined number of levels of the taxonomy (250) with the anchor sequence (760);
infer an embedding vector (732) from the language model (330) for each of the anchor sequence (760), the taxonomically similar sequence (770), and the taxonomically dissimilar sequence (780);
compute a triplet loss (804) by evaluating a triplet loss function (802) with the embedding vectors (732);
infer a taxonomy location prediction (742) from additional layers (340) of the classifier model (120) for each of the embedding vectors (732);
converting each expected taxonomy location (464) of the anchor sequence (760), the taxonomically similar sequence (770), and the taxonomically dissimilar sequence (780) to taxonomy location hash value (566);
generating a total cross-entropy loss (754) by adding the results of invoking a cross-entropy loss function (704) on each of the taxonomy location predictions (742) and corresponding taxonomy location hash value (566); and
training the classifier model (120) based on the total cross-entropy loss (754) and the triplet loss (804).

10. The computing device of claim 9, wherein the sequence string comprises a first sequence string, wherein the instructions further cause the one or more processors to:
infer a location within the taxonomy of a second sequence string by providing the second sequence string as input to the classifier model.

11. The computing device of claim 9, wherein the sequence string is encoded as a string of letters, wherein the instructions further cause the one or more processors to:
convert the sequence string to one or more numeric values using a byte paired encoding operation.

12. The computing device of claim 9, wherein the taxonomy comprises a hierarchy of classification levels and each expected taxonomy location is converted to a single number, and wherein each of the taxonomy location predictions is a single number.

13. A method of performing metagenomic analysis on an environmental sample, the method comprising:
collecting the environmental sample, the environmental sample comprising genomic material from a plurality of organisms;
generating sequence data from the genomic material, wherein the sequence data is not correlated with taxonomic categories of the plurality of organisms;
providing the sequence data to a machine learning model, the machine learning model trained with masking language modeling on a corpus of polynucleotide sequences and trained with triplet loss on a corpus of labeled polynucleotide sequences; and
receiving from the machine learning model a plurality of taxonomic classifications for the plurality of organisms.

14. The method of claim 13, wherein the sequence data comprises at least 1,000,000 unique reads.

15. The method of claim 13, wherein the environmental sample is collected from a body of water, soil, or the digestive tract of a vertebrate.

16. The method of claim 13, wherein the sequence data comprises sequences of 16S ribosomal RNA genes.

17. The method of claim 13, wherein the plurality of taxonomic classifications comprises at least one of, domain, kingdom, phylum, class, order, family, genus, or species.

18. The method of claim 13, wherein the machine learning model comprises an attention-based bi-directional transformer layer.

19. The method of claim 13, wherein the machine learning model is trained using a total loss function that sums cross-entropy loss and triplet loss.

20. The method of claim 13, further comprising:
generating two-dimensional representations of higher-dimensionality vectors representing the taxonomic classifications for the plurality of organisms; and
displaying the two-dimensional representations on a display device.
